(19) United States
(12) Patent Application Publication
(10) Pub. No.: US 2019/0090955 A1
(43) Pub. Date: Mar. 28, 2019

(54) SYSTEMS AND METHODS FOR POSITION AND ORIENTATION TRACKING OF ANATOMY AND SURGICAL INSTRUMENTS

(71) Applicant: MIRUS LLC, Atlanta, GA (US)

(72) Inventors: Angad SINGH, Atlanta, GA (US); Jay YADAV, Sandy Springs, GA (US)

(21) Appl. No.: 16/081,598

(22) PCT Filed: Mar. 1, 2017
(86) PCT No.: PCT/US17/20146

§ 371 Date: Aug. 31, 2018

Related U.S. Application Data
(60) Provisional application No. 62/301,736, filed on Mar. 1, 2016.
(60) Provisional application No. 62/359,259, filed on Jul. 7, 2016.
(60) Provisional application No. 62/394,955, filed on Sep. 15, 2016.
(60) Provisional application No. 62/394,962, filed on Sep. 15, 2016.
(60) Provisional application No. 62/395,343, filed on Sep. 15, 2016.

(51) Int. Cl. A61B 34/20; A61B 34/10; A61B 90/00

(52) U.S. Cl. CPC A61B 34/20; A61B 34/10; A61B 90/39; A61B 2090/3983; A61B 2090/3945; A61B 2034/104; A61B 2090/3916; A61B 2090/3962; A61B 2034/2055; A61B 2034/105; A61B 2034/2048

(57) ABSTRACT

Systems and methods are provided for estimating pose of an anatomy and pose of surgical instruments relative to the anatomy. The systems and/or methods can include registering a patient's actual anatomy. The systems and/or methods can further include receiving visual and sensory information indicative of pose of the anatomy and surgical instruments relative to the anatomy.

### CROSS REFERENCE TO RELATED APPLICATIONS

This application claims the benefit of U.S. Provisional Patent Application No. 62/301,736, filed on Mar. 1, 2016, entitled “FIDUCIAL MARKER HAVING AN ORIENTATION SENSOR MODULE,” U.S. Provisional Patent Application No. 62/359,259, filed on Jul. 7, 2016, entitled “SYSTEMS AND METHODS FOR POSITION AND ORIENTATION TRACKING OF ANATOMY AND SURGICAL INSTRUMENTS,” U.S. Provisional Patent Application No. 62/394,955, filed on Sep. 15, 2016, entitled “SYSTEMS AND METHODS FOR POSITION AND ORIENTATION TRACKING OF ANATOMY AND SURGICAL INSTRUMENTS,” U.S. Provisional Patent Application No. 62/394,962, filed on Sep. 15, 2016, entitled “SYSTEMS AND METHODS FOR POSITION AND ORIENTATION TRACKING OF ANATOMY AND SURGICAL INSTRUMENTS,” and U.S. Provisional Patent Application No. 62/395,343, filed on Sep. 15, 2016, entitled “SYSTEMS AND METHODS FOR POSITION AND ORIENTATION TRACKING OF ANATOMY AND SURGICAL INSTRUMENTS,” the disclosures of which are expressly incorporated herein by reference in their entireties.

### TECHNICAL FIELD

The present disclosure relates generally to orthopedic surgery including, but not limited to, joints, spine, upper and lower extremities, and maxillofacial surgery and, more particularly, to a system and method for intra-operative tracking of the position and orientation of the patient's anatomy, a surgical instrument, and/or a prosthesis used in the surgery.

### BACKGROUND

Many orthopedic surgeries, such as those involving the spine, are complex procedures that require a high degree of precision. For example, the spine is in close proximity to delicate anatomical structures such as the spinal cord and nerve roots. Compounding the problem is limited surgical exposure and visibility, particularly in the case of minimally invasive procedures. Consequently, the risk of misplaced implants or other complications is high.

Similarly, in orthopedic procedures involving resurfacing, replacement, or reconstruction of joints using multi component prosthesis with articulating surfaces, proper placement of the prosthetic component is critical for longevity of the implant, positive clinical outcomes, and patient satisfaction.

Currently, many orthopedic surgeons intra-operatively evaluate prosthetic component placement using an imprecise combination of subjective experience of the surgeon and rudimentary mechanical instrumentation. For example, in hip replacement surgery, there are three parameters that are typically used to quantify differences in prosthetic joint placement: leg length (also called hip length), offset, and anterior/posterior position. Leg length refers to the longitudinal extent of the leg measured in the superior/inferior axis relative to the pelvis. Offset refers to the position of the leg in the medial-lateral axis relative to the pelvis. Anterior/posterior (“AP”) position of the leg, as the name suggests, refers to position of the leg along the anterior/posterior axis with respect to the pelvis.

Early methods for calculating leg length, offset, and anterior/posterior position required the surgeon to use rulers and gauges to perform manual measurements on the hip joint before and after attaching the prosthetic implants. Such measurements, however, are often inaccurate due to the difficulty in performing manual measurements in the surgical environment using conventional rulers and gauges. Further, manual measurements are not easily repeatable or verifiable, and can take a significant amount of time to perform.

In surgeries involving complex anatomies, such as spine surgery, the surgeon may rely on intraoperative imaging to guide and assess the placement of prosthesis. However imaging is typically not real-time and has to be repeated whenever there is movement of the anatomy and/or surgical instrument thereby exposing the patient and surgical team to harmful radiation over the duration of the procedure.

Because existing techniques for intra-operative evaluation are extremely subjective and imprecise, the performance of the corrected anatomy is highly variable and dependent on the experience level of the surgeon. Perhaps not surprisingly, it is difficult for patients and doctors to reliably predict the relative success of the surgery (and the need for subsequent corrective/adjustment surgeries) until well after the initial procedure. Such uncertainty has a negative impact on long term clinical outcomes, patient quality of life, and the ability to predict and control costs associated with surgery, recovery, and rehabilitation.

Some computer/robotically-assisted surgical systems provide a platform for more reliably estimating prosthetic placement parameters. These systems typically require complex tracking equipment, bulky markers/sensors, time-consuming instrument calibration/registration procedures that have to be repeated during the procedure, and highly-specialized software packages that often require technical support personnel to work with doctor in the operating room. Not only do such systems tend to be costly, they also tend to be far too complex to warrant broad adoption among orthopedic surgeons. Additionally, image-guided systems require repeated intraoperative imaging (e.g. fluoroscopy, CT scan, etc) which subjects the patient and surgical team to high doses of radiation.

The presently disclosed system and associated methods for intra-operatively measuring position and orientation of the anatomy and surgical instruments are directed to overcoming one or more of the problems set forth above and/or other problems in the art.

### SUMMARY

According to one aspect, the present disclosure is directed to a method for estimating a pose (e.g., position and/or orientation) of an anatomy for real-time intra operative tracking and guidance. The pose is estimated by receiving information from a visual-inertial system comprising a camera-based vision system that tracks one or more fiducial markers attached to the anatomy and/or one or more inertial sensors (e.g., inertial measurement units) attached to the anatomy. As described herein, the fiducial marker can include the inertial sensor such that the fiducial marker with inertial sensor is attached to the same anatomy in some implementations. Alternatively, the fiducial marker can be separate from the inertial sensor in some implementations. In this case, the fiducial marker and inertial sensor can be attached to the same or different anatomy. The estimated pose is used to update clinically relevant parameters, path trajectories, surgical plan predictions, and/or a virtual anatomic models for real-time visualization of the surgery. The method further includes registration of the patient's anatomy involving receiving from vision system and/or inertial measurement units information indicative of one or more anatomic reference positions, axes, planes, landmarks, or surfaces.

In accordance with another aspect, the present disclosure is directed to a method for estimating a pose of a surgical instrument relative to a patient's anatomy. The method includes real-time tracking of one or more fiducial markers and/or one or more inertial sensors also attached to the surgical instrument and calculation of clinically-relevant position parameters and/or visualization of the surgical instrument and/or its pose by receiving information from the above described visual-inertial system. As described herein, the fiducial marker can include the inertial sensor such that the fiducial marker with inertial sensor is attached to the surgical instrument in some implementations. Alternatively, the fiducial marker can be separate from the inertial sensor in some implementations. In this case, the fiducial marker and inertial sensor can be separately attached to the surgical instrument.

In accordance with another aspect, the present disclosure is directed to a system for estimating a pose of an anatomy or surgical instrument relative to the anatomy. The system includes fiducial markers and/or inertial sensors coupled to a patient's anatomy and surgical instrument. The system also includes one or more imaging devices (e.g., cameras) close to the surgical field, such as mounted on the surgical table or the anatomy itself. Alternatively, the imaging devices may be integrated with surgical lighting or other surgical equipment such as imaging equipment (e.g., X-ray machine or other imaging equipment). The system also includes a processor, communicatively coupled to the inertial sensors and imaging devices. The processor may be configured to create a virtual multi dimensional model of the anatomy from 2D or 3D images (e.g., pre-operative and/or intra-operative images). The processor may also be configured to register one or more axes, planes, landmarks or surfaces associated with a patient's anatomy. The processor may be further configured to estimate the pose of the patient's anatomy during surgery and animate/visualize the virtual model in real-time without the need for additional imaging. The processor may be further configured to estimate geometrical relationship between a surgical instrument and the patient's anatomy.

The fiducial markers utilized in the system are visual and/or visual-inertial. For example, in some implementations, the fiducial markers are visual fiducial markers. In other implementation, the fiducial markers are combined visual-inertial fiducial markers, meaning inertial sensors are physically coupled to the fiducial marker. Visual refers to features or patterns that are recognizable by a camera or vision system and inertial refers to sensors that measure inertial data such as acceleration, gravity, angular velocity, etc. For example, the fiducial marker may include an inertial sensor and at least one patterned, reflective or light-emitting feature.

In some implementations, the fiducial marker includes planar two dimensional patterns or contoured surfaces. The contoured or patterned surface can aid an imaging system in recognizing the fiducial marker and determine pose of the fiducial marker from the projection of the contoured or patterned feature on the camera image plane. Such fiducial markers may be easily placed on any flat surface including on the patient's body. The pattern may encode information such as a bar code or QR code. Such information may include a unique identifier as a well as other information to facilitate localization.

Alternatively or additionally, in some implementations, the fiducial marker is a contoured or patterned three dimensional surface.

Alternatively or additionally, in some implementations, the fiducial marker includes a reflective surface. The reflective surface can aid an imaging system in recognizing the fiducial marker and determine pose of the fiducial marker from the projection of the reflective surface on the camera image plane.

Alternatively or additionally, in some implementations, the fiducial marker is a light source. Optionally, the light source can be a light-emitting diode. Alternatively or additionally, the light source can optionally be configured to emit light at a predetermined frequency, which can aid an imaging system in recognizing the fiducial marker and determine pose of the fiducial marker from the projection of the light source on the camera image plane. Alternatively or additionally, the light source can optionally be configured to emit light having a predetermined pattern, which can aid an imaging system in recognizing the fiducial marker.

In some implementations, the fiducial marker can optionally include a diffuser element. The diffuser element can be configured to condition reflected or emitted light. The diffuser element can be a textured glass or polymer housing the contains the entire fiducial marker or be arranged in proximity to or at least partially surrounding the fiducial marker.

In some implementations described herein, the inertial sensor is an inertial measurement unit including at least one of a gyroscope, an accelerometer, or a magnetometer. Optionally, the inertial measurement unit further includes a network module configured for communication over a network. For example, the network module can be configured for wireless communication.

The image capturing device (sometimes also referred to herein as “imaging device”) utilized in the system may be a visible light monocular or stereo camera (e.g., a red-green-blue (RGB) camera) of appropriate resolution and/or specific to one or more wavelengths of interest such as infrared. The image capturing device may also be equipped with multi-spectral imaging capabilities to allow simultaneous imaging at different wavelengths. The image capturing device may be communicatively coupled to the processing unit via a wired connection or wirelessly.

Alternatively or additionally, the image capturing device utilized in the system may be a depth camera providing depth information in addition to RGB information. The image capturing device may be communicatively coupled to the processing unit via a wired connection or wirelessly.

An example method for estimating a pose of an anatomy of a patient is described herein. The method can include establishing, via a registration process, first information indicative of an anatomic reference. For example, the anatomic reference can include one or more anatomic positions, axes, planes, landmarks, or surfaces. The method can also include receiving, via one or more inertial measurement units, second information indicative of a change in the pose of the anatomy; receiving, via one or more imaging devices, third information indicative of a change in the pose of the anatomy; and estimating an updated pose of the anatomy based on the first information, the second information, and the third information.

In some implementations, the method can include tracking a fiducial marker using the imaging device.

Alternatively or additionally, the fiducial marker can include a pattered or contoured surface.

Alternatively or additionally, the fiducial marker can include a light reflector or a light-emitting source.

In some implementations, the fiducial marker can optionally include one or more inertial measurement units. Additionally, the method can further include fusing the second information and the third information. The updated pose of the anatomy can be estimated based on the first information and the fused second and third information. Optionally, the second information and the third information are fused using a Kalman filter or an extended Kalman filter.

Alternatively or additionally, the inertial measurement unit can be at least one of a gyroscope or an accelerometer

In some implementations, the method can further include displaying an estimated angle or a position between a plurality of anatomic features.

In some implementations, the method can further include displaying an estimated angle between an anatomic feature and an anatomic axis or plane.

In some implementations, the method can further include creating a virtual anatomic model of the anatomy using pre-operative or intra-operative images. The updated pose can be displayed by animating the virtual anatomic model of the anatomy.

Alternatively or additionally, the anatomy can be a portion of an upper extremity of a patient. Alternatively or additionally, the anatomy can be a portion of a lower extremity of a patient.

An example method for estimating a pose of a surgical instrument relative to an anatomy of a patient can include establishing, via a registration process, first information indicative of an anatomic reference. For example, the anatomic reference can include one or more anatomic positions, axes, planes, landmarks, or surfaces. The method can also include receiving, via one or more inertial measurement units, second information indicative of a change in the pose of the surgical instrument relative to the anatomy; receiving, via one or more imaging devices, third information indicative of a change in the pose of the surgical instrument relative to the anatomy; and estimating an updated pose of the surgical instrument relative to the anatomy based on the first information, the second information, and the third information.

In some implementations, the method can include tracking a fiducial marker using the imaging device.

Alternatively or additionally, the fiducial marker can include a pattered or contoured surface.

Alternatively or additionally, the fiducial marker can include a light reflector or a light-emitting source.

In some implementations, the fiducial marker can optionally include one or more inertial measurement units. Additionally, the method can further include fusing the second information and the third information. The updated pose of the anatomy can be estimated based on the first information and the fused second and third information. Optionally, the second information and the third information are fused using a Kalman filter or an extended Kalman filter.

Alternatively or additionally, the inertial measurement unit can be at least one of a gyroscope or an accelerometer

In some implementations, the method can further include displaying an estimated angle or a position between a plurality of anatomic features.

In some implementations, the method can further include displaying an estimated angle between an anatomic feature and an anatomic axis or plane.

In some implementations, the method can further include creating a virtual anatomic model of the anatomy using pre-operative or intra-operative images. The updated pose of the surgical instrument can be displayed on the virtual anatomic model of the anatomy.

In some implementations, the method can further include creating a virtual model of the surgical instrument.

Alternatively or additionally, the anatomy can be a portion of an upper extremity of a patient. Alternatively or additionally, the anatomy can be a portion of a lower extremity of a patient.

An example system for estimating a pose of an anatomy a patient can include one more imaging devices (or image capturing devices); one or more fiducial markers coupled to the anatomy; one or more inertial measurement units coupled to the anatomy and configured to detect information indicative of the pose of the anatomy; and a processor communicatively coupled to the imaging devices and inertial measurement units. The processor can be configured to establish, via a registration process, first information indicative of an anatomic reference. For example, the anatomic reference can include one or more anatomic positions, axes, planes, landmarks, or surfaces. The processor can be further configured to receive, via the inertial measurement unit, second information indicative of a change in the pose of the anatomy; receive, via imaging device, third information indicative of a change in the pose of the anatomy; and estimate an updated pose of the anatomy based on the first information, the second information, and the third information.

An example system for estimating a pose of an anatomy of a patient and a pose of a surgical instrument can include one or more imaging devices (or image capturing devices); a first set of fiducial markers and inertial measurement units coupled to the anatomy; a second set of fiducial markers and inertial measurement units coupled to the surgical instrument; and a processor communicatively coupled to the imaging device and the inertial measurement units of the first and second sets. The inertial measurement units of the first set can be configured to detect information indicative of the pose of the anatomy, and the inertial measurement units of the second set can be configured to detect information indicative of the pose of the surgical instrument. The processor can be configured to establish, via a registration process, first information indicative of an anatomic reference. For example, the anatomic reference can include one or more anatomic positions, axes, planes, landmarks, or surfaces. The processor can be further configured to receive, via the inertial measurement units of the first set or the inertial measurement units of the second set, second information indicative a change of at least one of the pose of the anatomy or the pose of the surgical instrument; receive, via the imaging device, third information indicative a change of at least one of the pose of the anatomy or the pose of the surgical instrument; and estimate an updated pose of the surgical instrument relative to the anatomy based on the first information, the second information, and the third information.

In some implementations, the imaging device can be mounted on the anatomy. In other implementations, the imaging device can be mounted on a surgical table. Optionally, the imaging device can be integrated with a surgical light. Optionally, the imaging device can be integrated with imaging equipment (e.g., an X-ray machine).

An example robotic surgical system for guiding or performing surgery can include one or more robotic arms of one or more degrees of freedom fitted with a surgical instrument. The robotic arm is communicatively coupled to a processor. The processor can be configured to control the motion of the robotic arm and/or set bounds on the motion the arm. The processor can also be configured to establish, via a registration process, first information indicative of an anatomic reference. For example, the anatomic reference can include one or more anatomic positions, axes, planes, landmarks, or surfaces. The processor can be further configured to receive, via one or more inertial measurement units, second information indicative of a change in the pose of the anatomy; receive, via one or more imaging devices, third information indicative of a change in the pose of the anatomy; and estimate an updated pose of the anatomy based on the first information, the second information, and the third information. The processor can also be configured to estimate an updated position of the robotic arm and/or boundaries of motion. One or more fiducial markers can be attached to the anatomy, and the fiducial marker can be tracked using the imaging device. Additionally, the robotic surgical system can be configured to perform or assist with surgery of an orthopedic or spinal structure.

An example fiducial marker is also described herein. The example fiducial marker may include at least one inertial measurement unit and at least one reflective or light-emitting source.

In some implementations, the inertial measurement unit includes a housing. Optionally, the source is integrated with the housing. Alternatively or additionally, the source is attached to or extends from the housing.

Alternatively or additionally, in some implementations, the housing defines a contoured surface. The contoured surface can aid an imaging system in recognizing the fiducial marker. Alternatively or additionally, in some implementations, the housing includes a patterned surface. The patterned surface can aid an imaging system in recognizing the fiducial marker.

Alternatively or additionally, in some implementations, the source is a light source. Optionally, the light source can be a light-emitting diode. Alternatively or additionally, the light source can optionally be configured to emit light at a predetermined frequency, which can aid an imaging system in recognizing the fiducial marker. Alternatively or additionally, the light source can optionally be configured to emit light having a predetermined pattern, which can aid an imaging system in recognizing the fiducial marker.

In some implementations, the fiducial marker can optionally include a diffuser element. The diffuser element can be configured to condition reflected or emitted light. Optionally, the diffuser element can be a textured glass or polymer housing for enclosing or containing the entire source. Alternatively or additionally, the diffuser element can be arranged in proximity to or at least partially surrounding the source.

Alternatively or additionally, in some implementations, the fiducial marker includes a plurality of reflective or light-emitting sources. Optionally, the sources can be arranged in a fixed spatial relationship with respect to one another.

Alternatively or additionally, in some implementations, the inertial measurement unit includes at least one of a gyroscope, an accelerometer, or a magnetometer. Optionally, the inertial measurement unit further includes a network module configured for communication over a network. For example, the network module can be configured for wireless communication.

Alternatively or additionally, in some implementations, the fiducial marker includes at least one of a magnet or an acoustic transducer. Alternatively or additionally, in some implementations, the fiducial marker can include a photosensor (e.g., a light measuring device) such as a photodiode, for example.

Alternatively or additionally, in some implementations, the fiducial marker and inertial measurement unit includes an elongate pin. Optionally, the inertial measurement unit or the source can be attached to the elongate pin. Alternatively or additionally, the elongate pin can optionally have a tapered distal end. Alternatively or additionally, the elongate pin can optionally have a threaded distal end. The distal end can be configured to anchor the fiducial marker to another object such as a subject's bone or a surgical instrument, for example.

Alternatively or additionally, in some implementations, the fiducial marker can include a quick connect/disconnect element. The quick connect/disconnect element can be configured for coupling with a base plate, which can facilitate easy fixation and removal to a base plate. The base plate can be attached to the subject's bone using a surgical pin or screw.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A provides a diagrammatic view of an example system used to measure pose of patient's anatomy consistent with certain disclosed embodiments.

FIG. 1B provides a diagrammatic view of an alternate system used to measure pose of a patient's anatomy consistent with certain disclosed embodiments.

FIG. 2 provides a diagrammatic view of an example system used to measure pose of a surgical instrument in relation to the patient's anatomy consistent with certain disclosed embodiments.

FIG. 3 provides a schematic view of example components associated with a system used to measure pose of an anatomy and/or surgical instruments, such as that illustrated in FIGS. 1A, 1B, 2, and 10.

FIG. 4 provides a flow of an example method associated with a sensor system used to measure pose of an anatomy and/or surgical instrument.

FIG. 5 is a fiducial marker according to one example described herein.

FIG. 6 is a fiducial marker according to another example described herein.

FIG. 7 is a fiducial marker according to yet another example described herein.

FIG. 8 is a fiducial marker according to yet another example described herein.

FIG. 9A is a flowchart illustrating example operations for estimating a pose of an anatomy. FIG. 9B is a flow chart illustrating example operations for estimating a pose of a surgical instrument relative to an anatomy.

FIG. 10 provides a diagrammatic view of an example system including a robot used to guide or perform surgical procedures consistent with certain disclosed embodiments.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Systems and methods consistent with the embodiments disclosed herein are directed to a visual-inertial system to measure the pose of a patient's anatomy as well as the pose of surgical instruments relative to the patient's anatomy. As used herein, pose is defined as position (X,Y,Z) and/or orientation (pitch, yaw, roll) with respect to a coordinate frame. Certain exemplary embodiments minimize the need for “image-based guidance,” meaning that they do not rely on repeated intra-operative imaging (e.g., fluoroscopy, X-ray, or computed tomography (CT)) which can add time and cost to the procedure and subject the patient to unnecessary exposure to potentially harmful radiation.

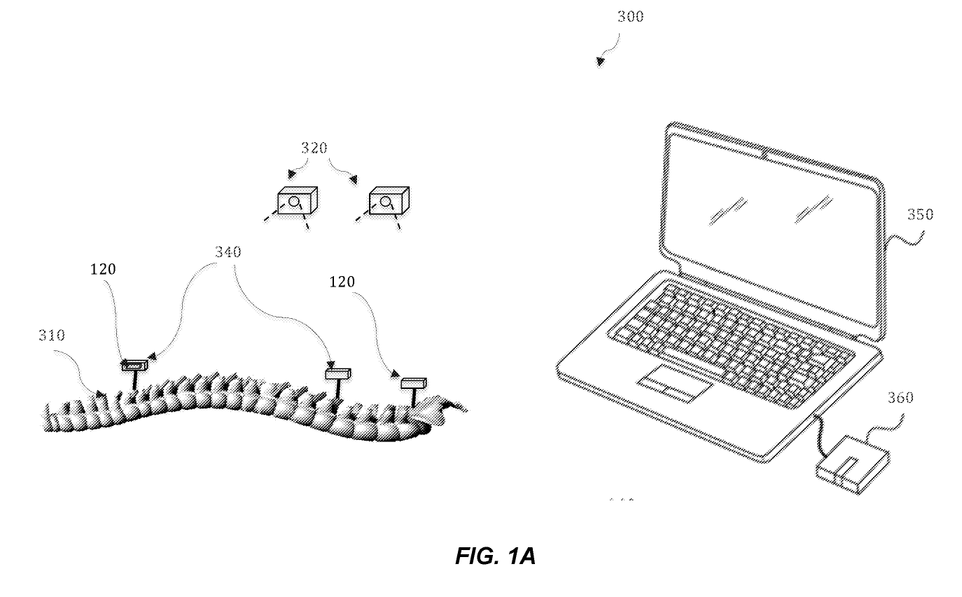

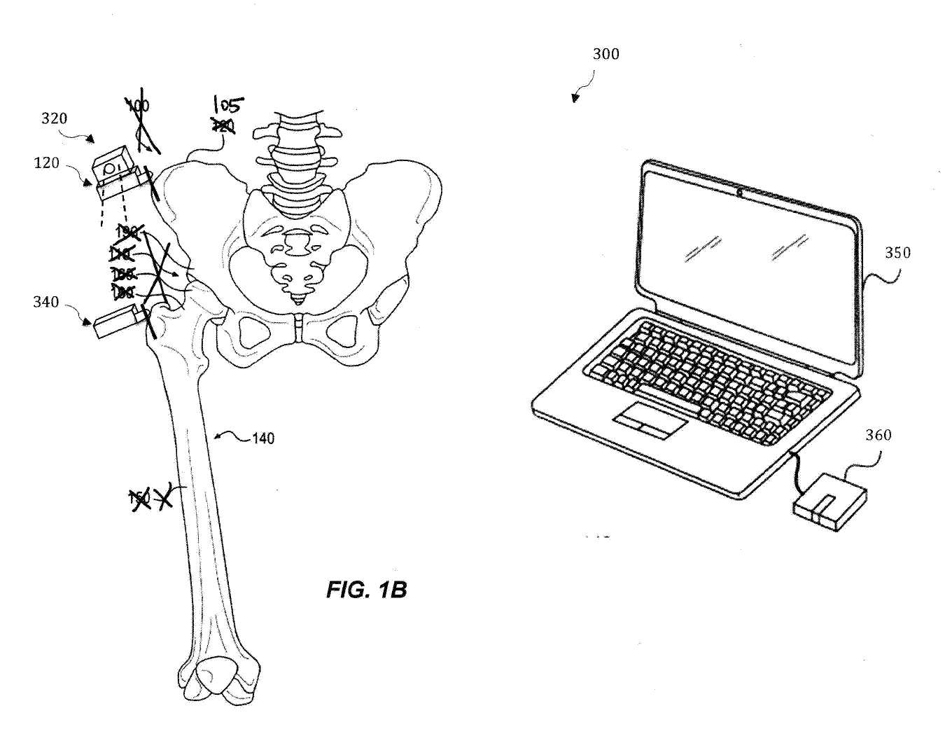

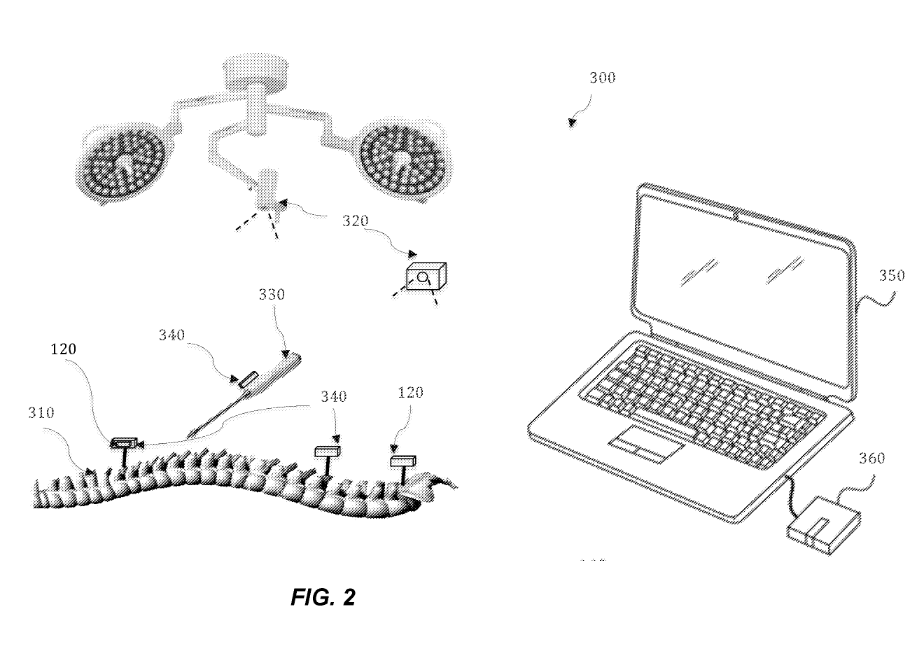

FIG. 1A provides a view depicting an example spine surgical system to measure the pose of a patient's spine. As illustrated in FIG. 1A, the surgical system 300 provides a solution for registering the spine 310, measuring the pose of the spine, and displaying this information in real-time. FIG. 1B provides a view depicting another example surgical system 300 to measure the pose of a patient's pelvis 105 and femur 140. As illustrated in FIG. 1B, the hip surgical system provides a solution for registering pelvic and/or femoral reference positions, axes and/or planes and measuring the changes in pose during and after the surgery and displaying this information in real-time. FIG. 2 provides a view depicting another example surgical system 300 to measure the pose of a surgical instrument 330 relative to a patient's spine 310. As illustrated in FIG. 2, in addition to the features of the system depicted in FIG. 1A, the spine surgical system provides a solution for registering the spine 310, measuring the pose of the surgical instrument 330 relative to the spine 310 and displaying this information in real-time. It should be understood that the spine and hip are only provided as examples of the patient's anatomy and that the systems and methods described herein are applicable to anatomy other than the spine or hip. For example, those skilled in the art will recognize that embodiments consistent with the presently disclosed systems and methods may be employed in any environment involving arthroplastic procedures, such as the knee and shoulder.

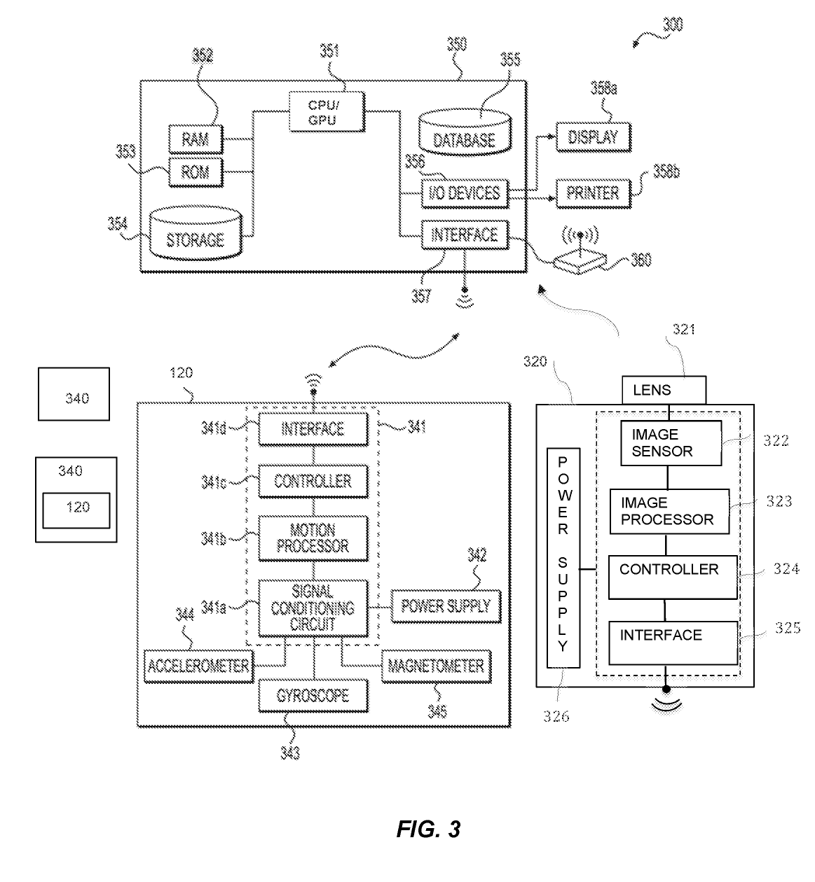

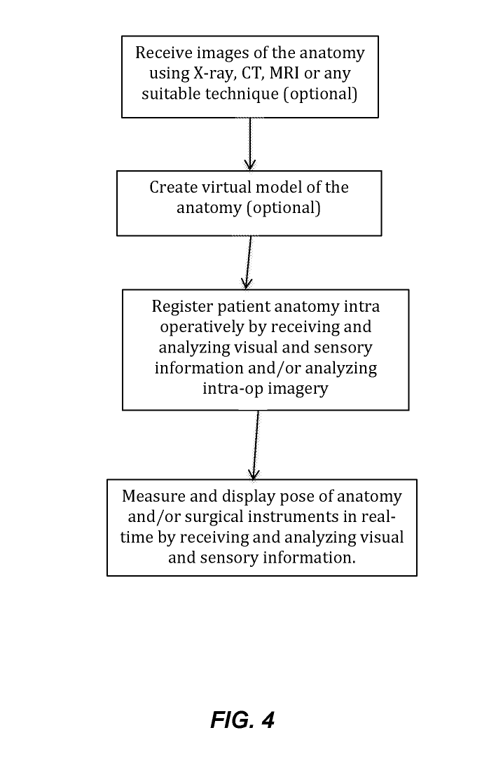

As illustrated in FIG. 1A, 1B, and 2, the system 300 comprises one or more fiducial markers 340, one or more inertial measurement units 120, and one or more imaging devices, for example, a camera 320 coupled to a processing and display unit 350. In some embodiments, wireless communication is achieved via wireless communication transceiver 360, which may be operatively connected to processing and display unit 350. Each fiducial marker 340 may contain a feature or features recognizable by the camera 320 and/or inertial sensors (e.g., inertial measurement unit 120 of FIG. 3) as described herein. Any number of fiducial markers and inertial sensors or any combination thereof can be placed on the anatomy depending on the application, number of anatomical segments to be independently tracked, desired resolution/accuracy of pose measurement, and type of information desired. For example, in FIGS. 1A and 2, one inertial measurement unit can be placed at the base of the spine 310. One fiducial marker 340 can be placed at the bottom of the thoracic spine and another fiducial marker 340 can be placed at the top of the thoracic spine. As shown in FIG. 1A, one or more of the fiducial markers 340 can include an inertial measurement unit 120. In other words, the visual fiducial marker can incorporate an inertial sensor. Other locations may be selected by the surgeon to achieve specific goals of the surgery. The system described herein facilitates the ability to miniaturize fiducial marker 340 and inertial measurement unit 120 such that they can be attached to small anatomical segments such as individual vertebrae. The fiducial markers and inertial sensors are placed on the anatomy using orthopedic screws or pins commonly used in such procedures. Alternatively, the fiducial markers and inertial sensors may be attached using custom clamps or quick connect/disconnect mechanisms or any means that ensures rigid fixation to the anatomy. The fiducial markers and inertial sensors can be placed on any suitable anatomical feature that allows for rigid fixation such as the spinous processes. Also, as illustrated in FIG. 2, fiducial marker 340 may be rigidly fixed on surgical instruments 330 at specified locations such that geometric relationship between fiducial marker 340 and the surgical instrument 330 is known. Alternatively, the system may determine the relative pose between the fiducial marker 340 and the surgical instrument 330 in real-time or via a registration process. Note that although there is no technical limitation on the number of fiducial markers that can be used, a practical limit is expected to be around 100 fiducials. However, the quantity of fiducial markers used does not interfere with or limit the disclosure in any way.

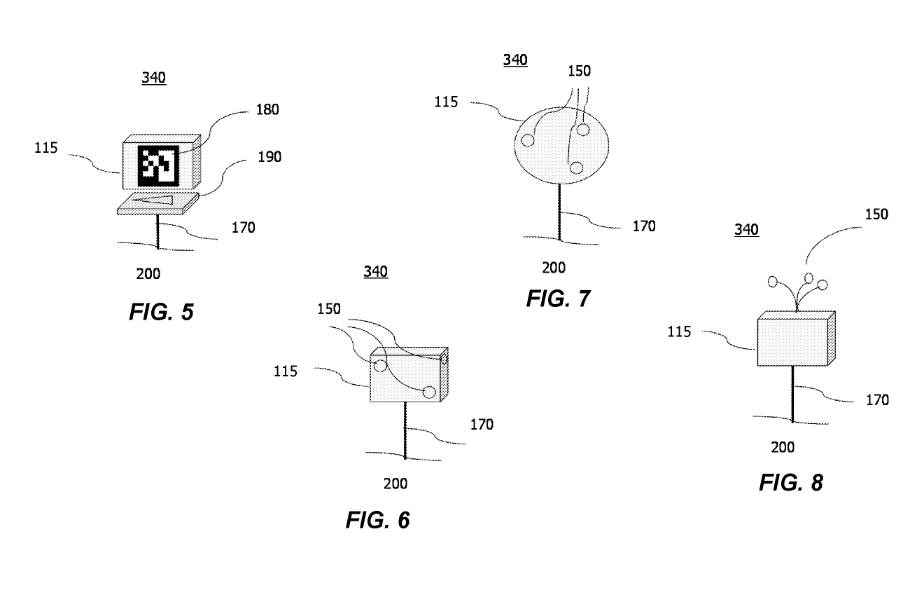

Referring now to FIGS. 5-8 example fiducial markers 340 according to implementations described herein are shown. In a general sense in the field of computer vision, a fiducial marker is a known object that can be easily identified. Therefore, there are numerous examples of two-dimensional (2D) (e.g., planar) and three-dimensional (3D) fiducial markers well known in the field and suitable for use in system shown in FIGS. 1A, 1B, and 2. FIGS. 5-8 are a few representative examples and should not be construed as limiting the disclosure in any way. Fiducial marker 340 as envisioned in the disclosed system can either be a purely visual marker containing visual features for localization and tracking by the camera-based vision system. Alternatively, fiducial marker 340 can optionally include inertial sensors (e.g., inertial measurement unit 120 described herein) in addition to the visual features. An example inertial measurement unit is described below (e.g., inertial measurement unit 120 of FIG. 3). As described below, the inertial measurement unit can be incorporated into a housing 115 of the fiducial marker.

In one embodiment, fiducial marker 340 contains a 2D or 3D patterned surface 180 (e.g., a checkered pattern, dot pattern, or other pattern) as shown in FIG. 5. The pattern can optionally be distinctive or conspicuous such that the patterned surface can aid an imaging system in recognizing the fiducial marker 340. The pattern can also encode a distinctive identifier and/or digital payload similar to a Quick Response (QR) code. Alternatively, the fiducial marker 340 contains a 2D or 3D contoured surface. The contoured surface can optionally be distinctive or conspicuous such that the surface can aid an imaging system in recognizing the fiducial marker 340.

In another embodiment, fiducial marker 340 can include of a reflective or light-emitting source 150 (referred to herein as “source(s) 150”). For example, each of the fiducial markers 340 of FIGS. 6-8 includes a plurality of sources 150 (e.g., 3 sources). It should be understood that FIGS. 6-8 are provided only as examples and that the fiducial marker 340 can include any number of sources 150. In addition, the sources 150 can be arranged in a fixed spatial relationship with respect to one another. The fixed spatial relationship can be distinctive or conspicuous such that the fiducial marker 340 can be recognized by the imaging system. The source 150 can be made of reflective material such that the source 150 reflects incident light. Alternatively or additionally, the source 150 can be a light source, e.g., a light-emitting diode or other light source. Additionally, the light source can optionally be configured to emit light at a predetermined frequency. Alternatively or additionally, the light source can optionally be configured to emit light having a predetermined pattern. It should be understood that providing emitted light with a predetermined frequency and/or pattern can aid an imaging system in recognizing and/or uniquely identifying the fiducial marker 340.

The fiducial marker 340 can include a housing 115. The housing 115 can enclose one or more components (described below) of the fiducial marker 340. Optionally, the source 150 can be integrated with the housing. For example, the source 150 can be integrated with an outer (e.g., exterior) surface of the housing 115 as shown in FIGS. 6-8. Alternatively or additionally, the source 150 can optionally be attached to or extend from the housing 115. For example, the source 150 can be attached to or extend from the outer surface of the housing 115 as shown in FIG. 8. Optionally, the housing 115 can define a patterned surface (e.g., a checkered pattern or other pattern) as discussed above with regard to FIG. 5. For example, at least a portion of the outer surface of the housing 115 can contain the pattern. Optionally, the housing 115 can include a contoured surface. For example, at least a portion of the outer surface of the housing 115 can be contoured. The contoured surface can optionally be distinctive or conspicuous such that the surface can aid an imaging system in recognizing the fiducial marker 340. It should be understood that the fiducial marker 340 shown in FIGS. 5-8 are provided only as examples and that the fiducial marker and/or its housing can be other shapes and/or sizes.

The fiducial marker 340 can include a quick connect feature such as a magnetic quick connect to allow for easy fixation to a base plate such as, for example, a base plate 190 shown in FIG. 5. The mating surface of the fiducial 340 and the base plate 190 may have a suitable keyed feature that ensure fixation of fiducial 340 to the base plate 190 in a fixed orientation and position.

The fiducial marker 340 or base plate 190 (if present) can include an elongate pin 170 as shown in FIG. 5-8. Alternatively or additionally, the elongate pin 170 can optionally have a tapered distal end. Alternatively or additionally, the elongate pin 170 can optionally have a threaded distal end. The distal end can be configured to anchor the fiducial marker 340 to another object 200 such as a subject's bone or a surgical instrument, for example.

Optionally, the fiducial marker 340 can include a diffuser element. The diffuser element can be configured to condition reflected or emitted light. For example, the diffuser element can be configured to diffuse or scatter reflected or emitted light. Optionally, the diffuser element can be a textured glass or polymer housing for enclosing or containing the source 150. The diffuser element can optionally be arranged in proximity to or at least partially surrounding the fiducial. Alternatively or additionally, the fiducial marker 340 can optionally include at least one of a magnetic field generator or an acoustic transducer. Alternatively or additionally, the fiducial marker 340 can include a photosensor (e.g., a light measuring device) such as a photodiode, for example.

As discussed herein, the fiducial marker 340 can optionally include inertial sensors such as, for example, inertial measurement unit 120 of FIG. 3. In this case, the housing 115 of the fiducial marker 340 can enclose one or more components (described below) of the inertial measurement unit 120. Depending on the embodiment of fiducial marker 340 as previously discussed, the respective visual features may be integrated within or on the housing 115. For example, a 2D or 3D patterned surface can be integrated with an outer (e.g., exterior) surface of the housing 115 as shown in FIG. 5. For example, the source 150 can be integrated with an outer (e.g., exterior) surface of the housing 115 as shown in FIGS. 6 and 7. Alternatively or additionally, the source 150 can optionally be attached to or extend from the housing 115 as shown in FIG. 8. It should be understood that FIGS. 5-8 are provided only as examples and that the housing 115 of fiducial marker 340 containing the inertial measurement unit 120 can be in other shapes and/or sizes.

Inertial measurement unit 120 may include one or more subcomponents configured to detect and transmit information that either represents the pose or can be used to derive the pose of any object that is affixed relative to inertial measurement unit 120, such as a patient's anatomy or surgical instrument.

According to one embodiment, inertial measurement unit 120 may include or embody one or more of gyroscopes and accelerometers. The inertial measurement unit 120 may also include magnetic sensors such as magnetometers. Inertial measurement units measure earth's gravity as well as linear and rotational motion that can be processed to calculate pose relative to a reference coordinate frame. Magnetic sensors measure the strength and/or direction of a magnetic field, for example the strength and direction of the earth's magnetic field or a magnetic field emanating from magnetic field generator. Using “sensor fusion” algorithms, some of which are well known in the art, the inertial measurement units and/or magnetic sensors may combine to measure full 6 degree-of-freedom (DOF) motion and pose relative to a reference coordinate frame. Inertial measurement unit 120 consistent with the disclosed embodiments is described in greater detail below with respect to the schematic diagram of FIG. 3.

Inertial measurement unit 120 associated with the presently disclosed system may each be configured to communicate wirelessly with each other and to a processing and display unit 350 that can be a laptop computer, PDA, or any portable, wearable (such as augmented/virtual reality glasses or headsets) or desktop computing device. The wireless communication can be achieved via any standard radio frequency communication protocol such Bluetooth, Wi Fi, ZigBee, etc., or a custom protocol. In some embodiments, wireless communication is achieved via wireless communication transceiver 360, which may be operatively connected to processing and display unit 350.

The processing and display unit 350 runs software that calculates the pose of the anatomy 310 and/or surgical instrument 330 based on the inertial and/or visual information and displays the information on a screen in a variety of ways based on surgeon preferences including overlaying of virtual information on real anatomic views as seen by the surgeon so as to create an augmented reality. The surgeon or surgical assistants can interact with the processing unit either via a keyboard, wired or wireless buttons, touch screens, voice activated commands, or any other technologies that currently exist or may be developed in the future.

In addition to their role as described above, fiducial marker 340 and/or inertial measurement units 120 also allow a means for the system to register anatomic axes, planes, surfaces, and/or features as described herein. Once registered, the anatomic reference can be used to measure the pose of the anatomy 310 as well as the pose of the surgical instruments 330 relative to the anatomy. As described herein, in some implementations, the fiducial marker 340 is purely a visual fiducial marker. Alternatively or additionally, in other implementations, the fiducial marker 340 can incorporate an inertial sensor such as inertial measurement unit 120. Optionally, inertial measurement unit 120 can be used for registration alone.

FIG. 3 provides a schematic diagram illustrating certain exemplary subsystems associated with system 300 and its constituent components. Specifically, FIG. 3 is a schematic block diagram depicting exemplary subcomponents of processing and display unit 350, fiducial marker 340, inertial measurement unit 120, and imaging device such as a camera 320. As described herein, this disclosure contemplates that the camera can be a monocular or stereo digital camera (e.g., RGB camera), depth camera, an infrared camera, and/or a multi-spectral imaging camera.

For example, in accordance with the exemplary embodiment illustrated in FIG. 3, system 300 may embody a system for intra-operatively—and in real-time or near real-time—measuring pose of an anatomy and/or surgical instrument. As illustrated in FIG. 3, system 300 may include a processing device (such as processing and display unit 350 (or other computer device for processing data received by system 300)), and one or more wireless communication transceivers 360 for communicating with the sensors attached to the patient's anatomy (not shown). The components of system 300 described above are examples only, and are not intended to be limiting. Indeed, it is contemplated that additional and/or different components may be included as part of system 300 without departing from the scope of the present disclosure. For example, although wireless communication transceiver 360 is illustrated as being a standalone device, it may be integrated within one or more other components, such as processing and display unit 350. Thus, the configuration and arrangement of components of system 300 illustrated in FIG. 3 are intended to be examples only.

Processing and display unit 350 may include or embody any suitable microprocessor-based device configured to process and/or analyze information indicative of the pose of an anatomy and/or surgical instrument. According to one embodiment, processing and display unit 350 may be a general purpose computer programmed with software for receiving, processing, and displaying information indicative of the pose of the anatomy and/or surgical instrument. According to other embodiments, processing and display unit 350 may be a special-purpose computer, specifically designed to communicate with, and process information for, other components associated with system 300. Individual components of, and processes/methods performed by, processing and display unit 350 will be discussed in more detail below.

Processing and display unit 350 may be communicatively coupled to the fiducial marker(s) 340, the inertial measurement unit(s) 120, and camera 320 and may be configured to receive, process, and/or analyze sensory and/or visual data measured by the fiducial marker 340 and/or camera 320. Processing and display unit 350 may also be configured to receive, process, and/or analyze sensory data measured by the inertial measurement unit 120. According to one embodiment, processing and display unit 350 may be wirelessly coupled to fiducial marker 340, the inertial measurement unit(s) 120, and camera 320 via wireless communication transceiver(s) 360 operating any suitable protocol for supporting wireless (e.g., wireless USB, ZigBee, Bluetooth, Wi-Fi, etc.) In accordance with another embodiment, processing and display unit 350 may be wirelessly coupled to fiducial marker 340, the inertial measurement unit(s) 120, and camera 320, which, in turn, may be configured to collect data from the other constituent sensors and deliver it to processing and display unit 350. In accordance with yet another embodiment, certain components of processing and display unit 350 (e.g. I/O devices 356) may be suitably miniaturized for integration with fiducial marker 340, the inertial measurement unit(s) 120, and camera 320.

Wireless communication transceiver(s) 360 may include any device suitable for supporting wireless communication between one or more components of system 300. As explained above, wireless communication transceiver(s) 360 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 360 may embody a standalone communication module, separate from processing and display unit 350. As such, wireless communication transceiver 360 may be electrically coupled to processing and display unit 350 via USB or other data communication link and configured to deliver data received therein to processing and display unit 350 for further processing/analysis. According to other embodiments, wireless communication transceiver 360 may embody an integrated wireless transceiver chipset, such as the Bluetooth, Wi-Fi, NFC, or 802.11x wireless chipset included as part of processing and display unit 350.

As explained, processing and display unit 350 may be any processor-based computing system that is configured to receive pose information associated with an anatomy or surgical instrument, store anatomic registration information, analyze the received information to extract data indicative of the pose of the surgical instrumentation with respect to the patient's anatomy, and output the extracted data in real-time or near real-time. Non-limiting examples of processing and display unit 350 include a desktop or notebook computer, a tablet device, a smartphone, wearable computers including augmented/virtual reality glasses or headsets, handheld computers, or any other suitable processor-based computing system.

For example, as illustrated in FIG. 3, processing and display unit 350 may include one or more hardware and/or software components configured to execute software programs, such as algorithms for tracking the pose of the anatomy and/or surgical instruments. This disclosure contemplates using any algorithm known in the art for tracking the pose of the anatomy and/or the surgical instrument. According to one embodiment, processing and display unit 350 may include one or more hardware components such as, for example, a central processing unit (CPU), Graphics processing unit (GPU), or microprocessor 351, a random access memory (RAM) module 352, a read-only memory (ROM) module 353, a memory or data storage module 354, a database 355, one or more input/output (I/O) devices 356, and an interface 357. Alternatively and/or additionally, processing and display unit 350 may include one or more software media components such as, for example, a computer-readable medium including computer-executable instructions for performing methods consistent with certain disclosed embodiments. It is contemplated that one or more of the hardware components listed above may be implemented using software. For example, storage 354 may include a software partition associated with one or more other hardware components of processing and display unit 350. Processing and display unit 350 may include additional, fewer, and/or different components than those listed above. It is understood that the components listed above are examples only and not intended to be limiting.

CPU/GPU 351 may include one or more processors, each configured to execute instructions and process data to perform one or more functions associated with processing and display unit 350. As illustrated in FIG. 3, CPU/GPU 351 may be communicatively coupled to RAM 352, ROM 353, storage 354, database 355, I/O devices 356, and interface 357. CPU/GPU 351 may be configured to execute sequences of computer program instructions to perform various processes, which will be described in detail below. The computer program instructions may be loaded into RAM 352 for execution by CPU/GPU 351.

RAM 352 and ROM 353 may each include one or more devices for storing information associated with an operation of processing and display unit 350 and/or CPU/GPU 351. For example, ROM 353 may include a memory device configured to access and store information associated with processing and display unit 350, including information for identifying, initializing, and monitoring the operation of one or more components and subsystems of processing and display unit 350. RAM 352 may include a memory device for storing data associated with one or more operations of CPU/GPU 351. For example, ROM 353 may load instructions into RAM 352 for execution by CPU/GPU 351.

Storage 354 may include any type of mass storage device configured to store information that CPU/GPU 351 may need to perform processes consistent with the disclosed embodiments. For example, storage 354 may include one or more magnetic and/or optical disk devices, such as hard drives, CD-ROMs, DVD-ROMs, or any other type of mass media device. Alternatively or additionally, storage 354 may include flash memory mass media storage or other semiconductor-based storage medium.

Database 355 may include one or more software and/or hardware components that cooperate to store, organize, sort, filter, and/or arrange data used by processing and display unit 350 and/or CPU/GPU 351. For example, database 355 may include historical data such as, for example, stored placement and pose data associated with surgical procedures. CPU/GPU 351 may access the information stored in database 355 to provide a comparison between previous surgeries and the current (i.e., real-time) surgery. CPU/GPU 351 may also analyze current and previous surgical parameters to identify trends in historical data. These trends may then be recorded and analyzed to allow the surgeon or other medical professional to compare the pose parameters with different prosthesis designs and patient demographics. It is contemplated that database 355 may store additional and/or different information than that listed above. It is also contemplated that the database could reside on the “cloud” and be accessed via an internet connection using interface 357.

I/O devices 356 may include one or more components configured to communicate information with a user associated with system 300. For example, I/O devices may include a console with an integrated keyboard and mouse to allow a user to input parameters associated with processing and display unit 350. I/O devices 356 may also include a display including a graphical user interface (GUI) for outputting information on a display monitor 358a. In certain embodiments, the I/O devices may be suitably miniaturized and integrated with fiducial marker 340, the inertial measurement unit(s) 120, or camera 320. I/O devices 356 may also include peripheral devices such as, for example, a printer 358b for printing information associated with processing and display unit 350, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 357 may include one or more components configured to transmit and receive data via a communication network, such as the Internet, a local area network, a workstation peer-to-peer network, a direct link network, a wireless network, or any other suitable communication platform. For example, interface 357 may include one or more modulators, demodulators, multiplexers, demultiplexers, network communication devices, wireless devices, antennas, modems, and any other type of device configured to enable data communication via a communication network. According to one embodiment, interface 357 may be coupled to or include wireless communication devices, such as a module or modules configured to transmit information wirelessly using Wi-Fi, Bluetooth, or cellular wireless protocols. Alternatively or additionally, interface 357 may be configured for coupling to one or more peripheral communication devices, such as wireless communication transceiver 360.

According to one embodiment, inertial measurement unit 120 may be an integrated unit including a microprocessor 341, a power supply 342, and one or more of a gyroscope 343, an accelerometer 344, or a magnetometer 345. According to one embodiment, inertial measurement unit may contain a 3-axis gyroscope 343, a 3-axis accelerometer 344, and a 3-axes magnetometer 345. It is contemplated, however, that fewer of these devices with fewer axes can be used without departing from the scope of the present disclosure. For example, according to one embodiment, inertial measurement unit 120 may include only a gyroscope and an accelerometer, the gyroscope for calculating the orientation based on the rate of rotation of the device, and the accelerometer for measuring earth's gravity and linear motion. The accelerometer may provide corrections to the rate of rotation information (based on errors introduced into the gyroscope because of device movements that are not rotational or errors due to biases and drifts). In other words, the accelerometer may be used to correct the orientation information collected by the gyroscope. Similarly, the magnetometer 345 can be utilized to measure a magnetic field and can be utilized to further correct gyroscope errors and also correct accelerometer errors. The use of redundant and complementary devices increases the resolution and accuracy of the pose information. The data streams from multiple sensors may be “fused” using appropriate sensor fusion and filtering techniques. An example of a technique that may be suitable for use with the systems and methods described herein is a Kalman Filter or Extended Kalman filter.

As illustrated in FIG. 3, microprocessor 341 of inertial measurement unit 120 may include different processing modules or cores, which may cooperate to perform various processing functions. For example, microprocessor 341 may include, among other things, an interface 341d, a controller 341c, a motion processor 341b, and signal conditioning circuitry 341a. Controller 341c may also be configured to control and receive conditioned and processed data from one or more of gyroscope 343, accelerometer 344, and magnetometer 345 and transmit the received data to one or more remote receivers. The data may be pre-conditioned via signal conditioning circuitry 341a, which includes amplifiers and analog-to-digital converters or any such circuits. The signals may be further processed by a motion processor 341b. Motion processor 341b may be programmed with “sensor fusion” algorithms as previously discussed (e.g., Kalman filter or extended Kalman filter) to collect and process data from different sensors to generate error corrected pose information. The orientation component of the pose information may be a mathematically represented as an orientation or rotation quaternion, euler angles, direction cosine matrix, rotation matrix of any such mathematical construct for representing orientation known in the art. Accordingly, controller 341c may be communicatively coupled (e.g., wirelessly via interface 341d as shown in FIG. 3, or using a wireline protocol) to, for example, processing and display unit 350 and may be configured to transmit the pose data received from one or more of gyroscope 343, accelerometer 344, and magnetometer 345 to processing and display unit 350, for further analysis.

Interface 341d may include one or more components configured to transmit and receive data via a communication network, such as the Internet, a local area network, a workstation peer-to-peer network, a direct link network, a wireless network, or any other suitable communication platform. For example, interface 341d may include one or more modulators, demodulators, multiplexers, demultiplexers, network communication devices, wireless devices, antennas, modems, and any other type of device configured to enable data communication via a communication network. According to one embodiment, interface 341d may be coupled to or include wireless communication devices, such as a module or modules configured to transmit information wirelessly using Wi-Fi or Bluetooth wireless protocols. As illustrated in FIG. 3, inertial measurement unit 120 may be powered by power supply 342, such as a battery, fuel cell, MEMs micro-generator, or any other suitable compact power supply.

Importantly, although microprocessor 341 of inertial measurement unit 120 is illustrated as containing a number of discrete modules, it is contemplated that such a configuration should not be construed as limiting. Indeed, microprocessor 341 may include additional, fewer, and/or different modules than those described above with respect to FIG. 3, without departing from the scope of the present disclosure. Furthermore, in other instances of the present disclosure that describe a microprocessor are contemplated as being capable of performing many of the same functions as microprocessor 341 of inertial measurement unit 120 (e.g., signal conditioning, wireless communications, etc.) even though such processes are not explicitly described with respect to microprocessor 341. Those skilled in the art will recognize that many microprocessors include additional functionality (e.g., digital signal processing functions, data encryption functions, etc.) that are not explicitly described here. Such lack of explicit disclosure should not be construed as limiting. To the contrary, it will be readily apparent to those skilled in the art that such functionality is inherent to processing functions of many modern microprocessors, including the ones described herein.

Microprocessor 341 may be configured to receive data from one or more of gyroscope 343, accelerometer 344, and magnetometer 345, and transmit the received data to one or more remote receivers. Accordingly, microprocessor 341 may be communicatively coupled (e.g., wirelessly (as shown in FIG. 3, or using a wireline protocol) to, for example, processing and display unit 350 and configured to transmit the orientation and position data received from one or more of gyroscope 343, accelerometer 344, and magnetometer 345 to processing and display unit 350, for further analysis. As illustrated in FIG. 3, microprocessor 341 may be powered by power supply 342, such as a battery, fuel cell, MEMs micro-generator, or any other suitable compact power supply.

As shown in FIGS. 1A, 1B, 2, and 10 system 300 may further comprise a vision system consisting of one or more cameras 320 that are communicatively coupled, either wirelessly or using a wireline protocol, to display unit 350 and be controlled by CPU/GPU 351. Camera 320 may be placed anywhere in close proximity to the surgery as along as fiducial markers of interest can be clearly imaged. For example, as shown in FIG. 1B, the camera 320 may be rigidly attached to the patient's anatomy. In another embodiment, the camera 320 may be rigidly attached to the surgical tables using clamps or other suitable means. In yet another embodiment, as shown in FIG. 2, camera 320 may be integrated with overhead surgical lighting or any other appropriate equipment in the operating room such as X-ray or other imaging equipment.

This disclosure contemplates that any commercially available high definition (HD) digital video cameras such as the Panasonic HX-A1 of Panasonic corp. of Kadoma, Japan can be used. As shown in FIG. 3, camera 320 may comprise components that are commonly found in digital cameras. For example, camera 320 may include a lens 321 that collects and focuses the light on to an image sensor 322. The image sensor 322 can be any of several off-the-shelf image complementary metal-oxide-semiconductor (CMOS) image sensor available such as the IMX104 by Sony Electronics. Optionally or additionally, one or more of camera 320 may be an infra-red camera or a camera at another wavelength or in some cases a multispectral camera in which case one or more of the image sensor 322 will be chosen for the appropriate wavelength(s) and/or combined with appropriate filters. The camera 320 may also comprise an image processor 323 that processes the image and compressed/encodes into a suitable format for transmission to display unit 350. The image processor 323 may also perform image processing functions such image segmentation and object recognition. It is anticipated that certain image processing will also be performed on the display unit 350 using CPU/GPU 351 and processing load-sharing between image processor 323 and CPU/GPU 351 will be optimized based of the needs of the particular application after considering performance factors such as power consumption and frame rate. A controller unit 324 may be a separate unit or integrated into processor 323 and performs the function of controlling the operation of camera 320 and receiving commands from CPU/GPU 351 in display unit 350 as well as sending messages to CPU/GPU 351.

In addition or alternatively, camera 320 may be one or more depth cameras such as a Time of flight (ToF) camera or a RGB-D camera. An RGB-D camera is an RGB camera that augments its image with depth information. Examples of such cameras such as the SWISS RANGER SR4000/4500 from MESA IMAGING of Zurich, Switzerland and CARMIN AND CAPRI series cameras from PRIMESENSE of Tel Aviv, Israel.

As shown in FIG. 3, camera 320 may also comprise interface 325 may include one or more components configured to transmit and receive data via a communication network, such as the Internet, a local area network, a workstation peer-to-peer network, a direct link network, a wireless network, or any other suitable communication platform. For example, interface 325 may include one or more modulators, demodulators, multiplexers, demultiplexers, network communication devices, wireless devices, antennas, modems, and any other type of device configured to enable data communication via a communication network. According to one embodiment, interface 325 may be coupled to or include wireless communication devices, such as a module or modules configured to transmit information wirelessly using Wi-Fi or Bluetooth wireless protocols.

As illustrated in FIG. 3, camera 320 may be powered by power supply 326, such as a battery, fuel cell, MEMs micro-generator, or any other suitable compact power supply. The camera 320 may also be powered by the display unit 350 using a wired connection.

It also anticipated that in certain embodiments of the camera 320, it can optionally comprise one or more inertial sensors (e.g., inertial measurement unit 120 as described herein) as shown in FIG. 1B. In such embodiments, several functional units such as power supply, processor, and interface units may be shared between camera 320 and inertial sensor.

The camera 320 in conjunction with display unit 350 forms a vision system capable of calculating and displaying the pose of an anatomy or surgical instrument. For example, the camera 320 takes video images of one or more fiducial marker 340. The pose information contained in the images (e.g., pose of the anatomy and/or surgical instrument) is sometimes referred to herein as “third information.” Each image frame is analyzed and processed using algorithms that detect and localize specific visual patterns of the fiducial marker 340 such as pattern 180 in FIG. 5 or light emitting/reflecting light sources 150 in FIGS. 6-8. The algorithms further analyze the projection of the pattern or the light reflecting/emitting sources on the image plane and calculate the pose of the fiducial marker 340 in the real-world coordinates (e.g., a reference coordinate system). This final calculation relies in part on the calibration of the camera 320 which is performed prior to use. An example algorithm that performs the above sequence of operations in real-time is the open source AprilTag library (https://april.eecs.umich.edu/software/apriltag.html). It should be understood that AprilTag is only one example algorithm for processing images to detect and localize visual patterns of fiducial markers in order to calculate pose and that other algorithms may be used with the systems and methods described herein.

Although the vision system is capable of determining pose of the anatomy and/or surgical instrument on its own, system 300 is capable of fusing vision and inertial based methods to determine pose with greater resolution, speed, and robustness than is possible with systems that rely on any one type of information. For example, the pose information contained in the images (e.g., the “third information”), which is analyzed/processed as described above to obtain the pose in a reference coordinate system, can be fused with the pose information detected by the inertial sensor. The pose information detected by the inertial sensor such as the inertial measurement unit (e.g., pose of the anatomy and/or surgical instrument) is sometimes referred to herein as “second information.” In other words, the data streams from the inertial modalities (e.g., gyroscope, accelerometer, and/or magnetometer) may be “fused” with the pose obtained from the visual system using appropriate fusion and filtering techniques. An example of a technique that may be suitable for use with the systems and methods described herein is a Kalman Filter or an Extended Kalman Filter.

As explained, in order for system 300 to accurately estimate changes in pose of the anatomy 310 and/or pose of the surgical instrument 330 relative to the anatomy, it must the register the patient's anatomy in the operating room (OR) to establish information indicative of anatomic reference positions, axes, planes, landmarks, or surfaces. This is sometimes referred to herein as an anatomic reference, which can be contained in the “first information” described herein. Anatomic registration is a process of establishing the above information so that all pose data is presented relative to a anatomic reference (e.g., an anatomic reference coordinate system) and is therefore anatomically correct. The virtual model may be constructed from pre-operative or intra-operative images such as CT scan, for example or may simply be a generic representative model of the anatomy of interest. This disclosure contemplates using any modelling algorithm known in the art to create the virtual anatomic model such as the segmentation and modeling techniques currently used to convert DICOM images acquired by CT or MRI to 3D models. This disclosure contemplates using any registration algorithm known in the art to register the patient's anatomy to the virtual model such as point pair matching, surface/object matching, palpation of anatomic landmarks, and processing of single plane or multi-plane intra-operative imaging. The above described anatomic registration and 3D modeling allows the system to convert the pose information as derived from the inertial sensors and vision system into the appropriate anatomically correct components and display it in an anatomically correct fashion. The term “virtual,” is used herein to refer to a plane, vector, or coordinate system that exists as a mathematical or algorithmic representation within a computer software program.

It should be appreciated that the logical operations described herein with respect to the various figures may be implemented (1) as a sequence of computer implemented acts or program modules (i.e., software) running on a computing device (e.g., as included in the system of FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

One example process for anatomic registration is by attaching fiducial marker 340 and/or inertial measurement unit 120 to an elongate registration tool or pointer and either pointing or aligning the tool to certain bony landmarks. For example, system 300 may be configured to measure orientation of fiducial marker 340 or inertial measurement unit 120 while they are removably attached to an elongate registration tool that is aligned to specific pelvic, cervical, and/or lumbar landmarks. Alternatively, system 300 may be configured to measure the position of the tip of a pointer to which fiducial marker 340 is removable attached as the pointer palpates certain bony landmarks such as the spinous processes or collects points to map certain bony surfaces. Using geometrical relationships associated between the anatomical landmarks and/or surfaces and pose of fiducial marker 340, a coordinate space that is representative of the anatomy can be derived.

Another example process for registration uses intraoperative images (such as fluoroscopic X-rays) taken at known planes (A-P or lateral), in some cases with identifiable reference markers on the anatomy, and then virtually deforms/reshapes the virtual model to match the images. In such methods, one or more fiducial marker 340 or inertial measurement unit 120 may be rigidly attached to the imaging equipment if pose information of the imaging equipment is required to achieve accurate registration.

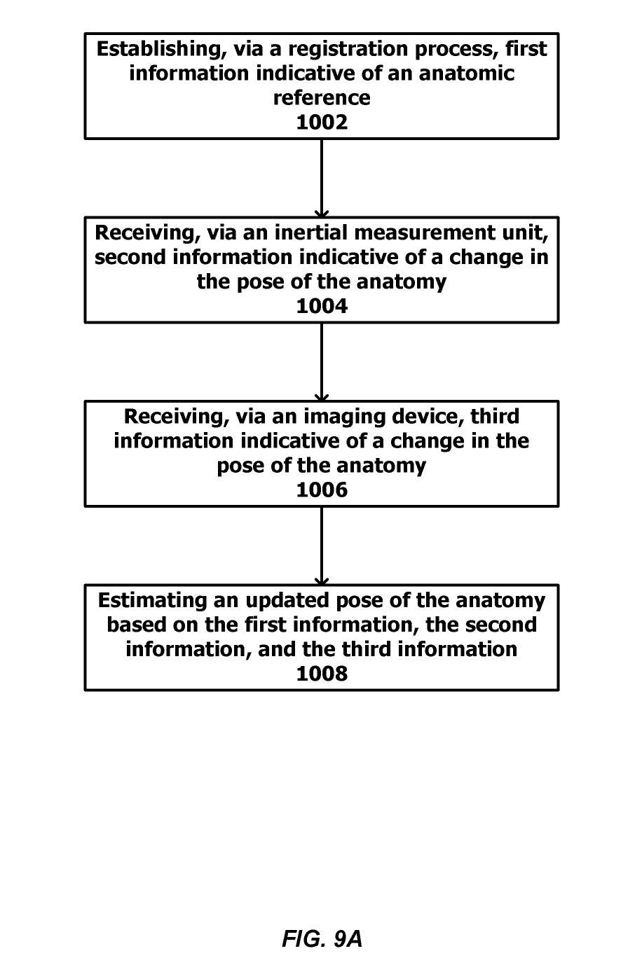

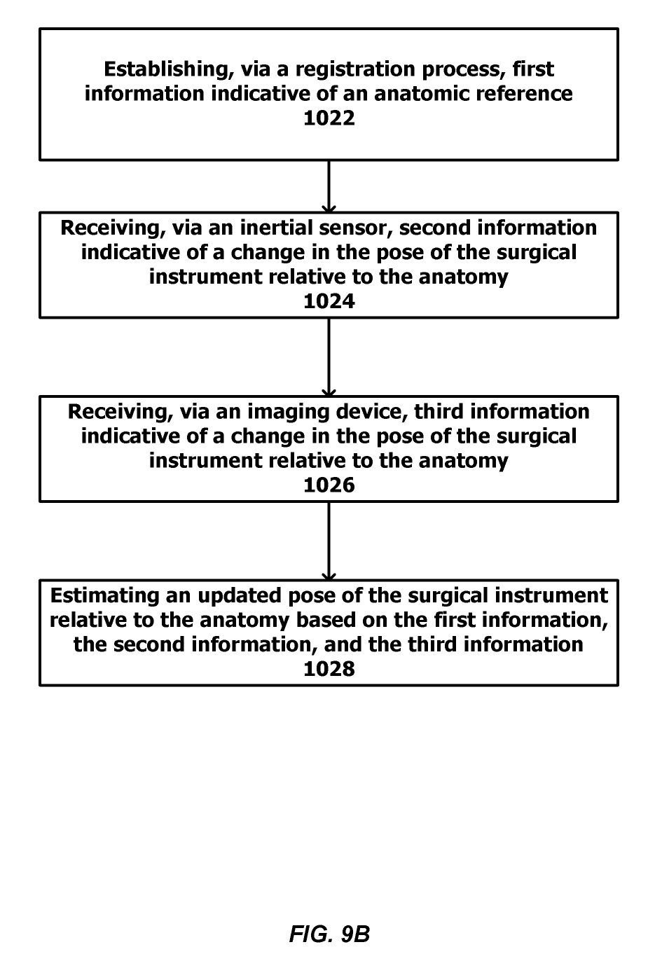

Referring now to FIG. 9A, an example method for estimating a pose of an anatomy is shown. This disclosure contemplates that this method can be performed using the example system described with regard to FIG. 3. At 1002, the method can include establishing, via a registration process, first information indicative of an anatomic reference. As described above, the patient's anatomy can be registered while in the operating room, for example, to establish anatomic reference positions, axes, planes, landmarks, or surfaces. This disclosure contemplates that the patient's anatomy can include, but is not limited to, the patient's spine, upper extremity (e.g., at least a portion of the arm), or lower extremity (e.g., at least a portion of the leg). At 1004, the method can also include receiving, via an inertial sensor (e.g., inertial measurement unit 120 of FIG. 3), second information indicative of a change in the pose of the anatomy. As described above, the second information includes data stream(s) output by the inertial sensor (e.g., gyroscope, accelerometer, and/or magnetometer). At 1006, the method can also include receiving, via an imaging device (e.g., camera 320 of FIG. 3), third information indicative of a change in the pose of the anatomy. As described above, the third information includes the pose information contained in the images. A change in the pose of the anatomy can be detected and localized by analyzing visual patterns of the fiducial marker(s) (e.g., fiducial marker 340 of FIGS. 5-8). For example, this disclosure contemplates analyzing the projections of a visual pattern of a fiducial marker on the imaging plane and calculating the pose of the fiducial marker therefrom. This calculated pose represents the pose of the anatomy to which the fiducial maker is fixed. As described herein, the fiducial marker can include the inertial sensors, i.e., the fiducial marker can incorporate an inertial measurement unit. In this case, the respective information indicative of the change in pose (e.g., information detected by the imaging device and information detected by the inertial measurement unit) can be fused, for example, using a Kalman filter or extended Kalman filter. At 1008, the method can also include estimating an updated pose of the anatomy based on the first information, the second information, and the third information.

Referring now to FIG. 9B, an example method for estimating a pose of a surgical instrument relative to an anatomy is shown. This disclosure contemplates that this method can be performed using the example system described with regard to FIG. 3. At 1022, the method can include establishing, via a registration process, first information indicative of an anatomic reference. As described above, the patient's anatomy can be registered while in the operating room, for example, to establish anatomic reference positions, axes, planes, landmarks, or surfaces. This disclosure contemplates that the patient's anatomy can include, but is not limited to, the patient's spine, upper extremity (e.g., at least a portion of the arm), or lower extremity (e.g., at least a portion of the leg). At 1024, the method can also include receiving, via an inertial sensor (e.g., inertial measuring unit 120 of FIG. 3), second information indicative of a change in the pose of the surgical instrument relative to the anatomy. As described above, the second information includes data stream(s) output by the inertial sensor (e.g., gyroscope, accelerometer, and/or magnetometer). At 1026, the method can also include receiving, via an imaging device (e.g., camera 320 of FIG. 3), third information indicative of a change in the pose of the surgical instrument relative to the anatomy. As described above, the third information includes the pose information contained in the images. A change in the pose of the anatomy and/or the surgical instrument can be detected and localized by analyzing visual patterns of the fiducial marker(s) (e.g., fiducial marker 340 of FIGS. 5-8). For example, this disclosure contemplates analyzing the projections of a visual pattern of a fiducial marker on the imaging plane and calculating the pose of the fiducial marker and/or surgical instrument therefrom. This calculated pose represents the pose of the anatomy and/or the surgical instrument to which the fiducial maker is fixed. As described herein, the fiducial marker can include the inertial measurement unit, i.e., the fiducial marker can incorporate the inertial measurement unit. In this case, the respective information indicative of the change in pose (e.g., information detected by the imaging device and information detected by the inertial measurement unit) can be fused, for example, using a Kalman filter or extended Kalman filter. At 1028, the method can also include estimating an updated pose of the surgical instrument relative to the anatomy based on the first information, the second information, and the third information.

In some implementations, the method can include tracking a fiducial marker using the imaging device.

In some implementations, the method can further include displaying an estimated angle or position between a plurality of anatomic features.

In some implementations, the method can further include displaying an estimated angle between an anatomic feature and an anatomic axis or plane.

In some implementations, the method can further include creating a virtual anatomic model of the anatomy using pre-operative or intra-operative images. The pose information can be displayed by animating the virtual anatomic model of the anatomy.

In some implementations, the method can further include creating a virtual model of the surgical instrument.

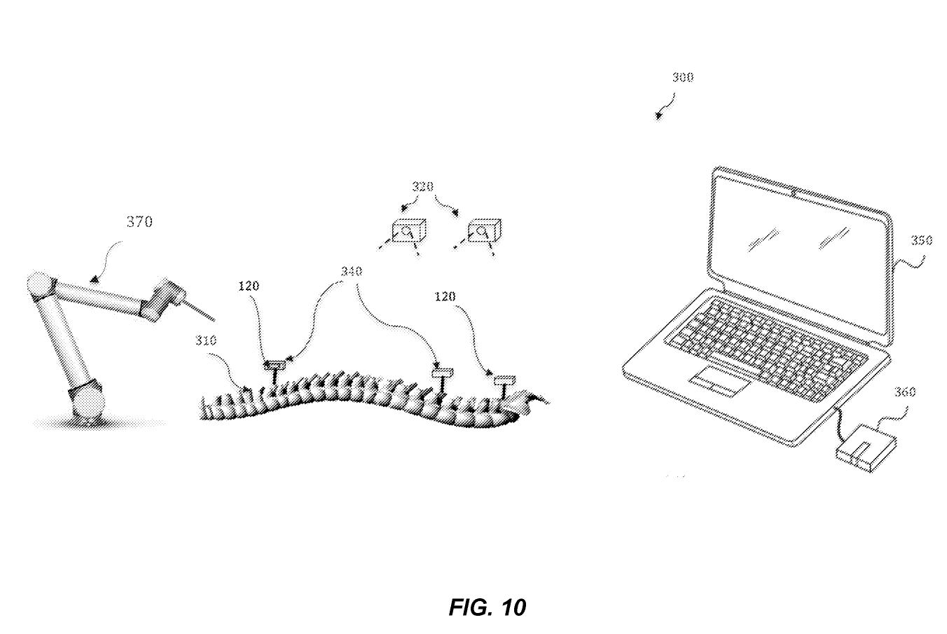

FIG. 10 provides a view depicting another example spine surgical system 300 to guide or perform surgical operations. As illustrated in FIG. 10, the spine surgical system 300 provides a solution for registering the spine 310, measuring the pose of the spine, and moving the robotic arm 370 to a desired position in relation to the anatomy. As described herein, the surgical system 300 can include processing and display unit 350 and wireless communication transceiver 360, which may be operatively connected to processing and display unit 350. This disclosure contemplates that the surgical system 300 can use one or more fiducial markers 340, one or more inertial measurement units 120, and one or more imaging devices, for example, a camera 320 coupled to a processing and display unit 350 to control the robotic arm 370 and/or estimate pose of the patient's anatomy (e.g., spine 310). It should be understood that the spine is only provided as an example of the patient's anatomy and that the systems and methods described herein are applicable to anatomy other than the spine, including but not limited to, a hip.

It will be apparent to those skilled in the art that various modifications and variations can be made to the disclosed systems and methods for measuring orientation and position of an anatomy or surgical instrument in orthopedic arthroplastic procedures. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

1. A method for estimating a pose of an anatomy of a patient or a surgical instrument, comprising:
establishing, via a registration process, first information indicative of an anatomic reference;
receiving, via a fiducial marker coupled to the anatomy or the surgical instrument, second information indicative of a change in the pose of the anatomy or the surgical instrument, wherein the fiducial marker comprises an inertial measurement unit;
receiving images of the fiducial marker coupled to the anatomy or the surgical instrument from an imaging device;
analyzing the images to obtain third information indicative of a change in the pose of the anatomy or the surgical instrument; and
estimating an updated pose of the anatomy or the surgical instrument based on the first information, the second information, and the third information.

2. (canceled)

3. The method of claim 1, wherein the fiducial marker comprises a patterned or contoured surface.

4. The method of claim 1, wherein the fiducial marker comprises a light reflector or a light-emitting source.

5. (canceled)

6. The method of claim 1, further comprising fusing the second information and the third information, wherein the updated pose of the anatomy or the surgical instrument is estimated based on the first information and the fused second and third information.

7. (canceled)

8. The method of claim 1, wherein the inertial measurement unit comprises at least one of a gyroscope or an accelerometer.

9. The method of claim 1, further comprising displaying an estimated angle or a position between a plurality of anatomic features axes, or planes.

10. (canceled)

11. The method of claim 1, further comprising creating a virtual model of the anatomy or the surgical instrument, and displaying the updated pose by animating the virtual model of the anatomy or the surgical instrument.

12-26. (canceled)

27. A system for estimating a pose of an anatomy of a patient or a surgical instrument, comprising:
an imaging device;
a fiducial marker coupled to the anatomy or the surgical instrument, wherein the fiducial marker comprises an inertial measurement unit configured to detect information indicative of the pose of the anatomy or the surgical instrument and
a processor communicatively coupled to the imaging device and the inertial measurement unit, the processor being configured to:
establish, via a registration process, first information indicative of an anatomic reference;
receive, via the inertial measurement unit, second information indicative of a change in the pose of the anatomy or the surgical instrument;
receive, via an imaging device, images of the fiducial marker coupled to the anatomy or the surgical instrument
analyze the images to obtain third information indicative of a change in the pose of the anatomy or the surgical instrument; and
estimate an updated pose of the anatomy or the surgical instrument based on the first information, the second information, and the third information.

28. (canceled)

29. The system of claim 27, wherein the processor is further configured to fuse the second information and the third information, wherein the updated pose of the anatomy or the surgical instrument is estimated based on the first information and the fused second and third information.

30. (canceled)

31. The system of claim 27, wherein the imaging device is mounted on the anatomy.

32. The system of claim 27, wherein the imaging device is mounted on a surgical table.

33. The system of claim 27, wherein the imaging device is integrated with a surgical light.

34-40. (canceled)

41. A fiducial marker, comprising:
an inertial measurement unit; and
at least one reflective or light-emitting source.

42-62. (canceled)

63. The method of claim 4, wherein the light-emitting source is configured to emit light at a predetermined frequency or having a predetermined pattern.

64. The method of claim 1, wherein the fiducial marker further comprises a light measuring device.

65. The method of claim 1, wherein the imaging device comprises an inertial measurement unit, the method further including receiving, via the inertial measurement unit of the imaging device, information indicative of a change in relative pose between the imaging device and the anatomy or the surgical instrument.

66. The method of claim 1, wherein the imaging device is a depth camera.

67. The method of claim 1, wherein the registration process comprises palpating bony landmarks or surfaces using a registration tool comprising a second fiducial marker, wherein the second fiducial marker comprises an inertial measurement unit.

68. The method of claim 1, wherein the registration process comprises using an intraoperative imager comprising a second fiducial marker, wherein the second fiducial marker comprises an inertial measurement unit.

69. The system of claim 27, wherein the imaging device comprises an inertial measurement unit, and wherein the processor is further configured to receive, via the inertial measurement unit of the imaging device, information indicative of a change in relative pose between the imaging device and the anatomy or the surgical instrument.

70. The system of claim 27, wherein the imaging device is integrated with a light source.
